(19) United States
(12) Patent Application Publication
(10) Pub. No.: US 2004/0243022 A1
(43) Pub. Date: Dec. 2, 2004

(54) Method and system of detecting vulnerable plaque by sensing motion of thin fibrous cap

(75) Inventors: James Carney, Eden Prairie, MN (US); Patrice Tremble, Santa Rosa, CA (US); Mark Brister, Encinitas, CA (US)

Correspondence Address: FRANK C. NICHOLAS, CARDINAL LAW GROUP, Suite 2000, 1603 Orrington Avenue, Evanston, IL, 60201, US

(73) Assignee: Medtronic Vascular, Inc., Santa Rosa, CA

(21) Appl. No.: 10/834,712

(22) Filed: Apr. 29, 2004

Related U.S. Application Data
(60) Provisional application No. 60/466,356, filed on Apr. 29, 2003.

(51) Int. Cl. A61B 5/00

(52) U.S. Cl. 600/552; 600/481; 600/407

(57) ABSTRACT

A method for detecting vulnerable plaque lesions in a blood vessel includes subjecting a localized area of the blood vessel to pressure pulses, while emitting electromagnetic radiation at the same localized area of the blood vessel. The pressure pulses cause vibrations of the vascular tissue and the vulnerable plaque lesions that are characteristic for each, which results in differing amounts of deflection of the electromagnetic radiation reflected from the vascular surface. The presence or absence of vulnerable plaque is determined from the extent of the deflection of the electromagnetic radiation reflected from the vascular surface. In another embodiment according to the invention, the presence or absence of vulnerable plaque lesions is determined from the intensity of vibrations of the vascular tissue in response to a pressure pulse.

### RELATED APPLICATION

This application claims priority to U.S. Provisional Application No. 60/466,356, “Method and System of Detecting Vulnerable Plaque by Sensing Motion of Thin Fibrous Cap” to James Carney et al., filed Apr. 29, 2003, the entirety of which is incorporated by reference.

### FIELD OF THE INVENTION

This invention relates generally to methods for detecting cardiac disease, and particularly to methods for detecting vulnerable plaque lesions in the vascular system.

### BACKGROUND OF THE INVENTION

Heart disease, specifically coronary artery disease, is a major cause of death, disability, and healthcare expense. Until recently, most heart disease was considered to be primarily the result of a progressive increase of hard plaque in the coronary arteries. This atherosclerotic disease process of hard plaques leads to a critical narrowing (stenosis) of the affected coronary artery and produces anginal syndromes, known commonly as chest pain. The progression of the narrowing reduces blood flow, triggering the formation of a blood clot. The clot may choke off the flow of oxygen rich blood (ischemia) to the heart muscles, causing a heart attack. Alternatively, the clot may break off and lodge in another organ vessel such as the brain, resulting in a thrombosis and stroke.

Within the past decade, evidence has emerged expanding the paradigm of atherosclerosis, coronary artery disease, and heart attacks. New clinical data suggests that the rupture of sometimes non-occlusive, vulnerable plaques causes the vast majority of heart attacks. The rate is estimated to be as high as 60-80 percent. In many instances vulnerable plaques do not impinge on the vessel lumen, rather, much like an abscess, they are ingrained under the arterial wall. For this reason, conventional angiography or fluoroscopy techniques are unlikely to detect the vulnerable plaque. Due to the difficulty associated with their detection and because angina is not typically produced, vulnerable plaques may be more dangerous than other plaques that cause pain.

The majority of vulnerable plaque lesions include a lipid pool, necrotic smooth muscle (endothelial) cells, and a dense infiltrate of macrophages contained by a thin fibrous cap, some of which are only two micrometers thick or less. The lipid pool is believed to be formed as a result of a pathological process involving low density lipoprotein (LDL), macrophages, and the inflammatory process. The macrophages oxidize the LDL producing foam cells. The macrophages, foam cells, and associated endothelial cells release various substances, such as tumor necrosis factor, tissue factor, and matrix proteinases. These substances can result in generalized cell necrosis and apoptosis, pro-coagulation, and weakening of the fibrous cap. The inflammation process may weaken the fibrous cap to the extent that sufficient mechanical stress, such as that produced by increased blood pressure, may result in rupture. The lipid core and other contents of the vulnerable plaque (emboli) may then spill into the blood stream thereby initiating a clotting cascade. The cascade produces a blood clot (thrombosis) that potentially results in a heart attack and/or stroke. The process is exacerbated due to the release of collagen and other plaque components (e.g., tissue factor), which enhance clotting upon their release.

Several strategies have been developed for the detection of vulnerable plaques involving the measurement of temperature within a blood vessel. Vulnerable plaque tissue temperature is generally elevated compared to healthy vascular tissue, and measurement of this temperature discrepancy may allow detection of the vulnerable plaque. However, the temperature of the vulnerable plaque is generally less than two degrees Celsius above that of the surrounding vascular tissue, and is frequently only about 0.5 degree Celsius above that of the surrounding tissue. Consequently, to accurately determine whether vulnerable plaque is present, it is necessary to thermally map the vascular area, a process that requires multiple measurements to be made using highly sensitive thermal detection. U.S. Pat. No. 6,514,214 discloses devices for making such measurements comprising a catheter with multiple temperature sensors that measure the temperature of either blood flowing within close proximity to the vessel wall, or of the wall itself. U.S. Pat. No. 6,245,026 discloses thermal mapping catheters capable of measuring the temperatures of thermal plaque surfaces and comparing them to the temperatures of the surrounding arterial walls. Because thermal sensors must be placed on, or very near the vessel wall, thermal sensing devices present a risk of injuring vascular tissue while obtaining the necessary temperature measurements.

Another detection strategy involves labeling vulnerable plaque lesions with a marker. The marker substance may be specific for a component and/or characteristic of the vulnerable plaque. For example, the marker may have an affinity for the vulnerable plaque, more so than for healthy tissue. Detection of the marker may thus allow detection of the vulnerable plaque. Alternatively, the marker may not necessarily have an affinity for the vulnerable plaque, but will simply change properties while associated with the vulnerable plaque. The property change may be detected and thus allow detection of the vulnerable plaque.

Direct imaging of the vulnerable plaque would provide a new approach to detection of vulnerable plaque. However, imaging vulnerable plaque is difficult due to the opacity of the vulnerable plaque through the blood stream. The flow of blood in the vicinity of the vulnerable plaque renders conventional direct imaging technologies using visible light impossible. U.S. Pat. No. 6,475,210 discloses a device that may emit and detect any one of infrared radiation, ultrasound radiation, or laser light. The emitted energy is directed at the vessel wall, and the reflected energy from the wall is detected by the device. Although the energy reflected by vulnerable plaque is sufficiently different from that reflected by healthy vascular tissue to permit detection of vulnerable plaque, the method may not be adequately sensitive to detect early stages of pathology.

Accordingly, it would be desirable to provide a highly sensitive method for detecting vulnerable plaque that would overcome the aforementioned and other disadvantages.

### SUMMARY OF THE INVENTION

One aspect according to the invention provides a method for detecting vulnerable plaque lesions in a blood vessel. The method includes generating a pressure pulse in a localized area of a blood vessel, while sending electromagnetic radiation directed at the inner surface of the vascular tissue in the same localized area of the blood vessel. The method further includes detecting a resulting electromagnetic radiation reflected from the vascular tissue surface while the vascular tissue surface is being deformed by the pressure pulse. The method further includes determining the presence of vulnerable plaque, based on the deflection of the electromagnetic radiation received from the vascular surface due to tissue deformation.

Another aspect according to the invention provides a method for detecting vulnerable plaque that includes generating a pressure pulse toward vascular tissue, and detecting a resulting vibration from an interaction of the pressure pulse with the vascular tissue. The method further includes determining vulnerable plaque lesions in the vascular tissue based on the resulting vibration.

Another aspect according to the invention provides a system for detecting vulnerable plaque lesions in a blood vessel comprising a catheter having: a pressure pulse generator, an electromagnetic radiation emitter, and an electromagnetic radiation detector, all operably coupled to the catheter. The components of the system are arranged so that the emitter emits electromagnetic radiation toward vascular tissue while the tissue is being deformed by the pressure pulse. The electromagnetic radiation detector receives the electromagnetic radiation reflected from the vascular tissue subjected to the pressure pulse. Changes in the reflected electromagnetic radiation, due to deflection caused by the movement of the vascular tissue, are detected by the electromagnetic radiation sensor. The presence of vulnerable plaque is determined from the changes in the reflected electromagnetic radiation detected by the electromagnetic radiation detector.

Another aspect according to the invention provides a system for detecting vulnerable plaque in a blood vessel comprising a catheter having a vibration sensing device coupled to the catheter. The vibration-sensing device is able to receive vibrations from the tissue of the vascular wall.

### BRIEF DESCRIPTION OF THE DRAWINGS

FIG. 1A is a side view of a device for detecting vulnerable plaque, the device having a pressure pulse generator, an optical fiber electromagnetic radiation emitter, and an electromagnetic radiation sensor. The electromagnetic radiation from the emitter is directed toward healthy vascular tissues and is reflected toward the electromagnetic sensor from healthy vascular tissue.

FIG. 1B is a diagrammatic representation of the signal of the electromagnetic radiation sensor when the electromagnetic radiation sensor is receiving electromagnetic radiation reflected from healthy vascular tissue.

FIG. 2A is a side view of a device for detecting vulnerable plaque, the device having a pressure pulse generator, an optical fiber electromagnetic radiation emitter, and an electromagnetic radiation sensor. The electromagnetic radiation from the emitter is directed toward vulnerable plaque, and is being reflected toward the electromagnetic radiation sensor from the vulnerable plaque.

FIG. 2B is a diagrammatic representation of the signal of the electromagnetic radiation sensor when the electromagnetic radiation sensor is receiving electromagnetic radiation reflected from the surface of a vulnerable plaque lesion.

FIG. 3A is a side view of a device for detecting vulnerable plaque, the device having a pressure pulse generator, an optical fiber electromagnetic radiation emitter and an array of three electromagnetic radiation sensors. The electromagnetic radiation from the transmitter is directed toward a vulnerable plaque lesion, and is being reflected toward the three electromagnetic radiation sensors from the vulnerable plaque.

FIG. 3B is a diagrammatic representation of the signal of each of the three electromagnetic radiation sensors, when the sensors are receiving electromagnetic radiation reflected from a vulnerable plaque lesion.

FIG. 4A is a side view of a device for detecting vulnerable plaque lesions, the device having a pressure pulse generator, an optical fiber electromagnetic radiation emitter and an array of three sensors. The sensors have a narrow angle of acceptance so that the position of the vulnerable plaque can be determined in relation to the sensors.

FIG. 4B is a diagrammatic representation of the signal of each of the three electromagnetic radiation sensors, when the sensors are receiving radiation reflected from vulnerable plaque. Because of their low acceptance angle, the sensors receive energy from the vulnerable plaque only when the vulnerable plaque is at or near a 90-degree angle to the sensors.

FIG. 5A is a side view of a device for detecting vulnerable plaque, the device having a pressure pulse generator and two vibration sensors. The vibration sensors are connected to feeler wires that contact the wall of the blood vessel including vulnerable plaque lesions, if present.

FIG. 5B is a diagrammatic representation of the signal for each of the vibration detectors when contacting either healthy vascular tissue or vulnerable plaque.

FIG. 6 is a flow diagram of a method of detecting vulnerable plaque lesions in a blood vessel in accordance with the present invention. The method of detection comprises comparing changes in the amount of electromagnetic radiation reflected from the surface of a vascular wall due to deflection of the electromagnetic radiation, caused by the movement of either the normal vascular tissue or the vulnerable plaque lesions in response to pressure pulses.

FIG. 7 is a flow diagram of a method of detecting vulnerable plaque lesions in a blood vessel in accordance with the present invention. The method of detection comprises comparing vibrations of either the vascular tissue or the vulnerable plaque lesions in response to a pressure pulse.

### DETAILED DESCRIPTION

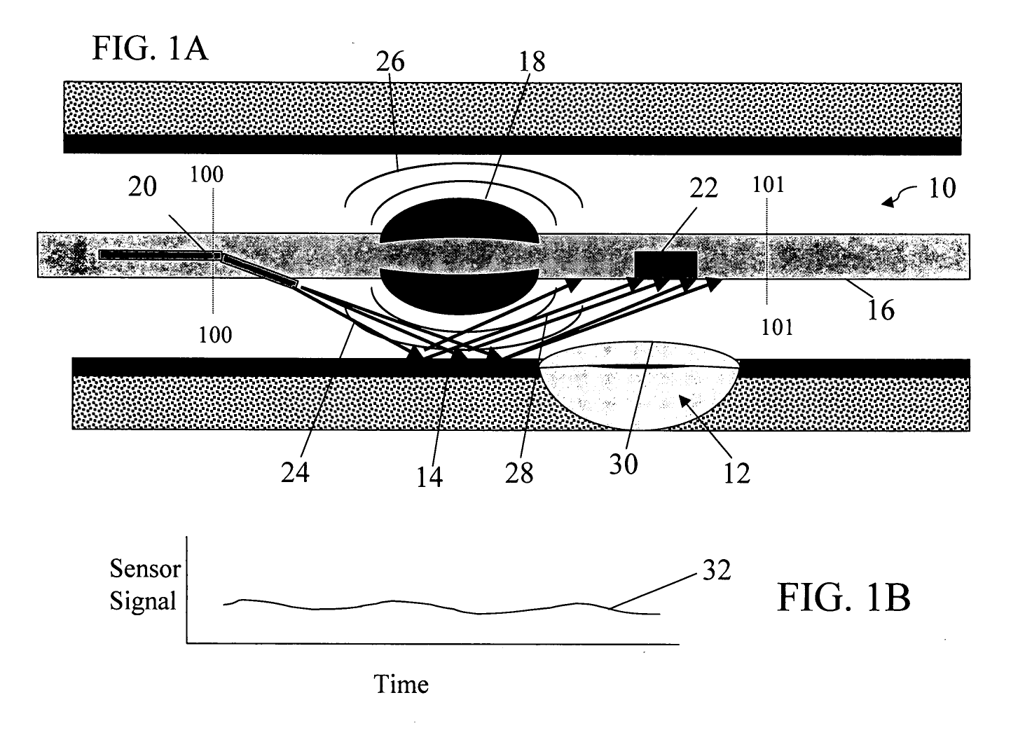

Referring to the drawings, FIG. 1A is a side view of a section of device 10 for detecting vulnerable plaque, according to the present invention. The device 10 is comprised of a catheter body 16 configured so that it can be inserted into the vascular system of a patient. Such catheter bodies are well known in the art and are typically made of flexible, biocompatible, polymeric materials such as polyurethane, polyethylene, nylon and polytetrafluroethylene (PTFE). Located near the distal end of catheter body 16 are an electromagnetic radiation emitter 20 and an electromagnetic radiation sensor 22. The electromagnetic radiation emitter 20 and sensor 22 are configured so that electromagnetic radiation emitted by emitter 20 will be reflected from the vascular wall 14 or vulnerable plaque lesion 30 and will be received by sensor 22. In order to minimize variability in the signal produced by the sensor, the dimensional relationship between the emitter 20 and sensor 22 must remain constant. Therefore, the segment of the catheter body between 100-100 and 101-101, that contains emitter 20 and sensor 22 is fairly rigid. The rest of the catheter body 16 may be flexible so that the catheter can be threaded through the vascular system of the patient with minimum risk of damaging the vessel walls.

In one embodiment, the electromagnetic radiation emitter 20 is connected to an electromagnetic radiation source by optical fibers that run longitudinally through the catheter and transmit the electromagnetic radiation from the source to emitter 20. In another embodiment, emitter 20 is an electromagnetic radiation emitting diode or diode laser. Emitter 20 is oriented so that the emitted electromagnetic radiation 24 is directed at an angle from the catheter and impinges on the vessel wall 14. The electromagnetic radiation may be of any wavelength that can be conducted through optical fibers, cables, or waveguides, or can be generated locally in the distal region of the catheter body 16 such as infrared, ultraviolet, visible, x-ray, beta, fluorescent, microwave or radio wave radiation. Laser light or ultrasonic energy may also be used. For optimal sensitivity, however, the electromagnetic radiation will be of a frequency that is absorbed or scattered minimally by the blood, but is reflected from the surface of the vascular tissue. For this reason, the preferred electromagnetic radiation is infrared radiation having a wavelength between 1550 and 1820 nanometers. The emitted infrared radiation 24 passes through the blood and impinges on a region of the vessel wall 14. The infrared radiation then is reflected from the vessel wall 14 toward the catheter body 16. Sensor 22 is positioned on the catheter body 16 so that the reflected infrared radiation 28 impinges on the sensor 22.

Numerous sensors for receiving electromagnetic radiation are known in the art and may be adapted for use in the present invention. The sensor 22 must be able to receive the electromagnetic radiation emitted by emitter 20, and will, therefore, generally be an infrared radiation sensor. The sensor 22 may be operably coupled to a fiber optic element that can conduct a signal from the diagnostic site to a site external to the patient. Alternatively, a radio wave or ultrasound transmitter may be used to transmit the signal from the sensor 22 to a site external to the patient.

In one embodiment, a pressure pulse generator 18 is positioned on the catheter body 16 in close proximity to the electromagnetic radiation emitter 20 and sensor 22. The pressure pulse generator 18 may be a piezoelectric element that acquires a charge when compressed or distorted and provides a transducer effect between electrical and mechanical oscillations. The piezoelectric element may consist of quartz, barium titanate, lead zirconate, lead titanate, lead zirconate titanate, a polymer such as polyvinylidene fluoride (PVDF) or ceramic. Alternatively, the pressure pulse generator 18 may be an electrostatic element that moves when a voltage is applied across it. Pressure pulse generator 18 may also be a balloon that can be inflated by forcing fluid through a lumen in the catheter and into the balloon. Pressure pulse generator 18 creates a localized pressure pulse 26 that causes the vessel wall 14 in close proximity to the pressure pulse generator 18 to distend slightly. In order to distend the vessel wall without risk of physiologic damage to the vessel wall, the pressure pulse would be controlled in the range of 80 to 180 mm Hg. The movement of the vessel wall 14 causes a deflection of the electromagnetic radiation 28 reflected from the wall. This deflection causes a momentary decrease in the electromagnetic radiation 28 received by the sensor 22. As shown in FIG. 1B, the signal produced by sensor 22 will have a slight wave-like pattern in response to the variations in electromagnetic radiation 28 received by the sensor 22. The wave-like pattern will have the same frequency as the frequency of the pressure pulse generator 18.

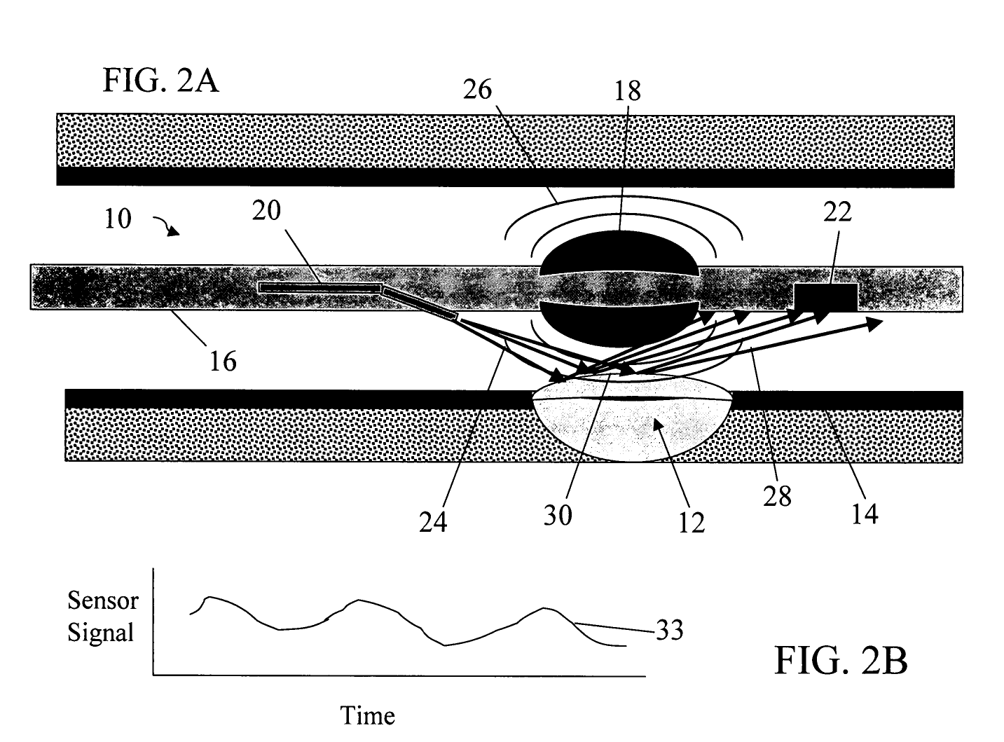

The majority of vulnerable plaque lesions include a lipid pool with a thin fibrous cap over the lesion. As a consequence, the mechanical properties of the vulnerable plaque lesion are very different from those of the vessel wall. Because of the fluid nature of the lipid pool, the vulnerable plaque lesion will be considerably more flexible than the vessel wall. As shown in FIG. 2A, the pressure pulse 26 will cause a much more pronounced deformation of the surface 30 of the vulnerable plaque lesion 12 than of the vessel wall 14. This greater deformation will, in turn, cause a greater deflection of the reflected electromagnetic radiation 28, resulting in greater variations in the electromagnetic radiation received by sensor 22. As indicated in FIG. 2B, the signal 33 from sensor 22 will have a more pronounced wave-like pattern than the signal 32 obtained from the vessel wall 14, and will be readily distinguishable from it, thus allowing diagnosis of a vulnerable plaque lesion 12 at that site in the vessel wall 14.

In one embodiment, a pressure pulse generator is not used. In this embodiment, the pressure pulse is generated by the pressure produced by the contractions of the patient's heart. The normal contractions of the heart generate a sufficient pressure pulse throughout the vasculature to cause deformation of the vessel wall 14 and deflection of the reflected electromagnetic radiation 28.

In another embodiment, a pulse generator 18 is used, as is shown in FIGS. 1A and 2A. In order to enhance the sensitivity of the device 10, the generation of the pressure pulse 26 and the measurements of the deflection of the reflected electromagnetic radiation 28 are timed so that the measurements are made between contractions of the heart. In this way, possible artifacts in the measurements due to normal movement of the heart and vasculature can be avoided. The pressure pulse generator 18 is synchronized with the contractions of the heart by attaching an electrode to the heart coupling the electrode to the device 10 so that the pressure pulse is triggered subsequent to the QRS electrical wave in the heart. When the R-wave, triggering the contraction of the left ventricle is detected, the device 10 is turned off for 300 to 400 msec while the heart contracts and blood is expelled from the left ventricle. After the 300 to 400 msec time period elapses, the device 10 is turned on until the next R-wave is detected.

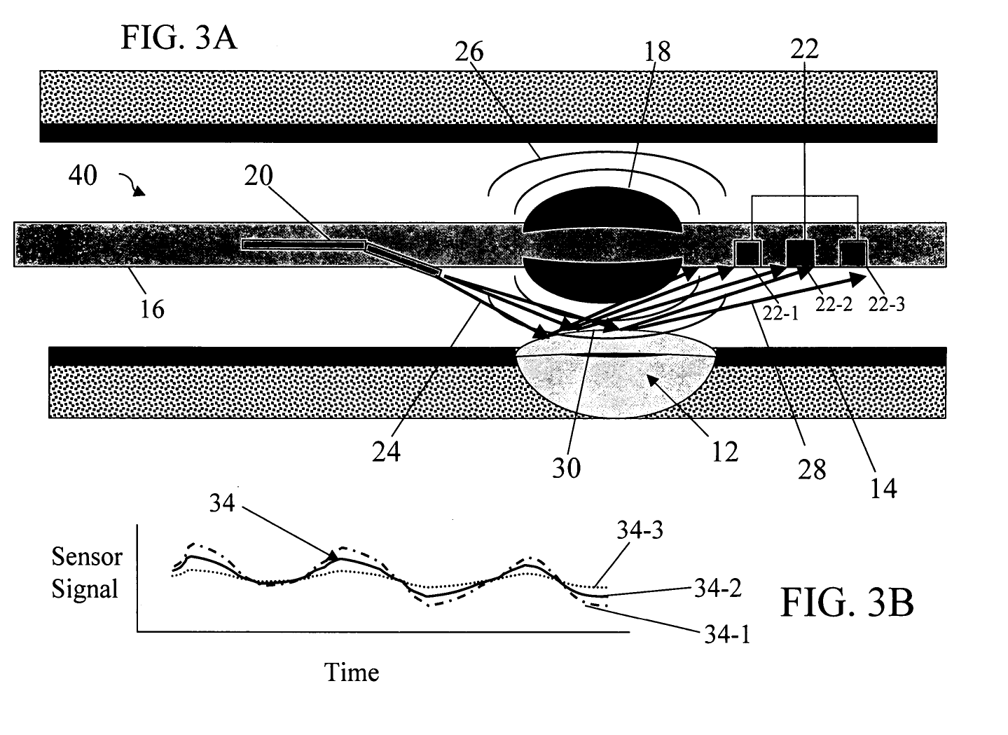

In another embodiment, the electromagnetic radiation sensor 22 of device 40 is comprised of an array of sensors, 22-1, 22-2, and 22-3, as shown in FIG. 3A. This configuration allows the array of sensors to receive more of the deflected electromagnetic radiation and as a result, enhances the sensitivity of the device. As shown in FIG. 3B, the sensor signal 34 is comprised of signals 34-1, 34-2 and 34-3 received from each sensor 22-1, 22-2, and 22-3, respectively. Each signal (34-1,34-2, and 34-3) can be recorded separately and thus provide multiple measurements of the deflection of the reflected electromagnetic radiation from each area of the vessel examined, resulting in greater accuracy than could be obtained using a single sensor.

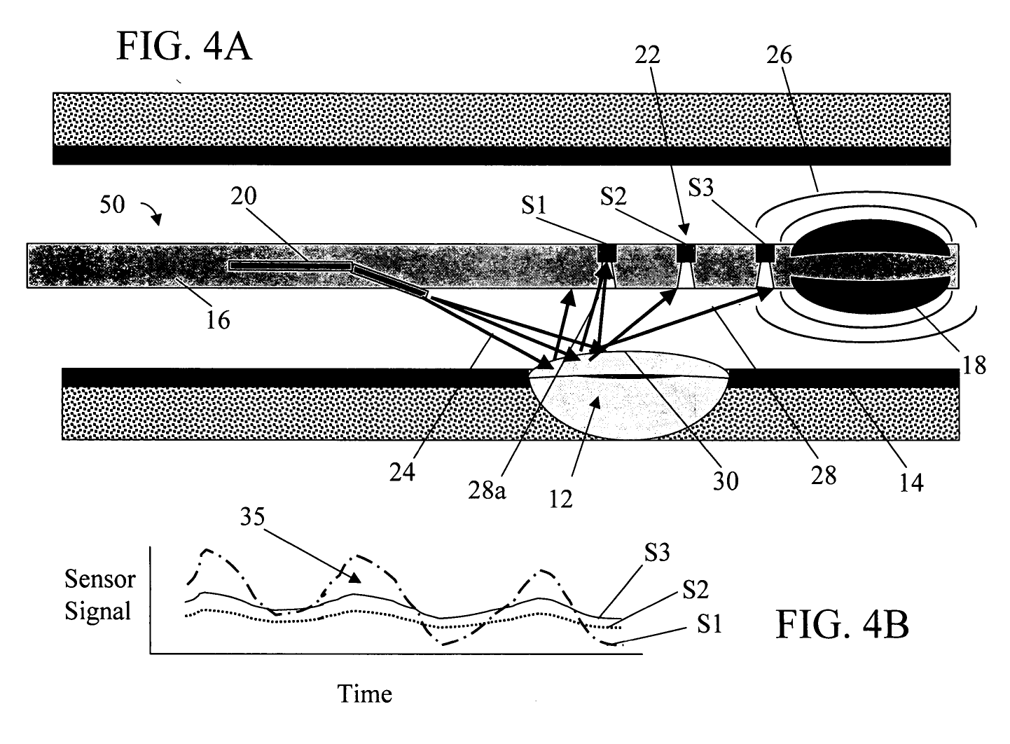

FIG. 4A represents another embodiment of device 50 according to the invention in which sensor 22 is comprised of an array of electromagnetic radiation sensors S1, S2, and S3, and the sensors have a narrow angle of acceptance so that only reflected electromagnetic radiation 28 that is nearly perpendicular 28a to the catheter body 14 is received by each sensor, S1, S2, or S3. The angle of acceptance may be controlled by recessing the sensors into the catheter body 14, or by placing a shield around the openings through the catheter body wall that provide access to the sensors (S1, S2, S3). Alternatively a lens may be placed between the sensor and the opening in the catheter body 14. The optimal acceptance cone in this embodiment according to the invention is centered about a 90 degree angle to the longitudinal axis of the catheter body. The acceptance angle of the cone may be adjusted according to the size of the blood vessel in order to accurately determine the location of the lesion. For large diameter blood vessels, the cone may have an acceptance angle of 10 degrees; for small diameter blood vessels the cone may have an acceptance angle of 30 degrees. By using an angle of acceptance in this range, only the vulnerable plaque lesions 12 immediately adjacent to the sensors (S1, S2, S3) will be detected. In this way, the location of the vulnerable plaque lesions 12 can be determined in relation to the sensors (S1, S2, S3) to within a millimeter or less.

FIG. 4B represents the signal 35 obtained from each sensor, S1, S2, and S3 of the sensor array 22 as the catheter body moves past a vulnerable plaque lesion 12. As shown in FIG. 4A, the vulnerable plaque lesion 12 is located at approximately a 90 degree angle to sensor S-1, but is at a smaller angle (approximately 30 to 45 degrees) to sensors S-2 and S-3. Only sensor S-1 receives a significant amount 28a of electromagnetic radiation 28 reflected from the vulnerable plaque lesion 12. This reflected electromagnetic radiation 28 exhibits the high deflection characteristic of vulnerable plaque lesions, and results in a sharp wave-like pattern in the signal SS-1 produced by sensor S-1. In comparison, there is very little deflection in the electromagnetic radiation received by sensors S-2 and S-3, and only a slight wave-like pattern in the sensor signals SS-1 and SS-2.

In yet another embodiment, an expandable member may be attached to the catheter body for the purpose of stabilizing the catheter within the blood vessel. The expandable member may be an inflatable balloon, an expandable wire mesh, an expandable spring, or any other means that may be used to reduce the movement of the catheter body and thereby reduce the variability in the measurements being made.

In another embodiment, a drug or drugs may be administered in order to dilate or contract the blood vessel being examined. The drug(s) may be administered singly or in combination, locally through the catheter, or systemically. Drugs that may be useful for this purpose include beta blockers, angiotensin, ACE inhibitors, vasodilators, and nitroglycerin. The drug(s) will cause the blood vessel lumen size to either increase or decrease, which in turn, will cause the fibrous caps of the vulnerable plaque lesions to be distended or contracted, and place the fibrous caps under greater or less tension. The changes in the fibrous caps would likely be greater than concomitant changes in the vessel wall. In this way, a drug regimen may be selected that will enhance the differences in the mechanical properties of the vulnerable plaque lesions compared the vessel wall and allow detection of small lesions characteristic of the early stages of disease.

In yet another embodiment, electromagnetic radiation may be both emitted and detected by a transceiver coupled to the catheter. This arrangement would allow detection and localization of vulnerable plaque lesions at approximately a 90 degree angle to the catheter body without a separate electromagnetic radiation emitter and sensor.

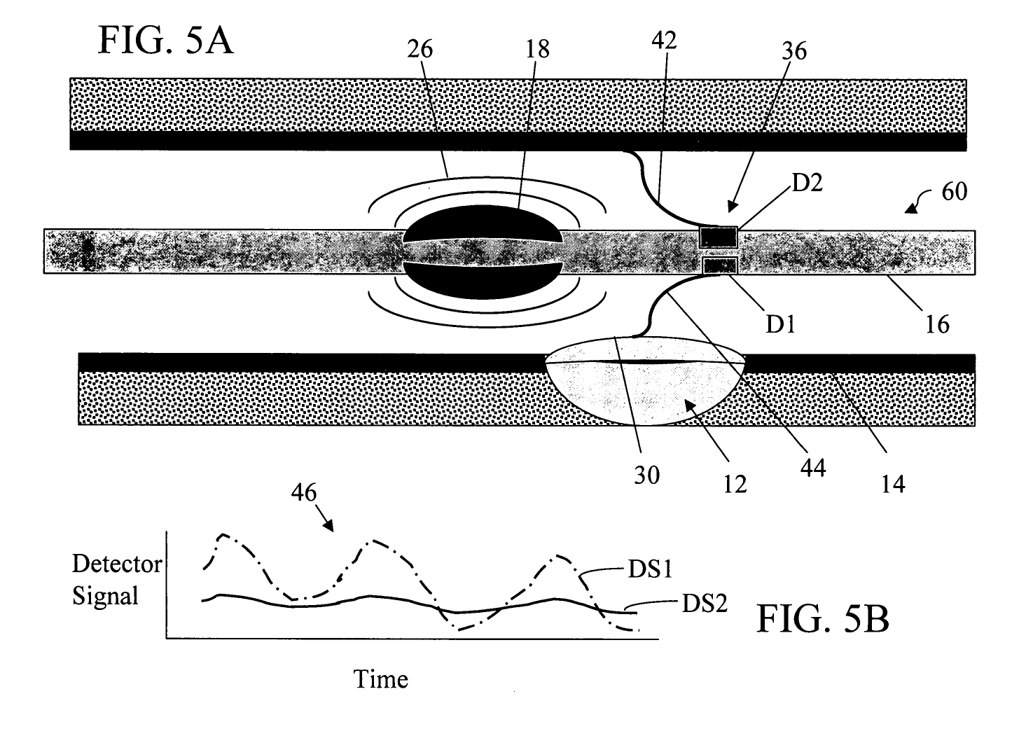

FIG. 5A is a side view of a device 60 for detecting vulnerable plaque 12 in a blood vessel in yet another embodiment according to the present invention. Pressure pulse generator 18 produces a localized pressure pulse 26 of sufficient magnitude to cause at least a slight distortion of the vessel wall 14. A vibration detector 36 coupled to the catheter body 14 detects the vibrations caused by the pressure pulse and produces a corresponding signal. Vibration detector 36 comprises at least two individual vibration sensors D1 and D2, but a plurality of vibration sensors may be used. In one embodiment, the vibration sensors are coupled to feeler wires 42 and 44 that touch the vascular wall 14, and as the catheter is advanced through the vasculature, the feeler wires are dragged along the vascular wall 14. As the pressure pulses cause the vessel wall to be distorted, the feeler wires 42 and 44 detect the movement as vibration, and transmit the information to the vibration sensors D1 and D2. Because of the fluid nature of the lipid pool in the vulnerable plaque lesions 12, the vibrations produced at the surface 30 of vulnerable plaque lesions 12 will be significantly larger than those produced in the vascular wall 14.

As indicated in FIG. 5A, the feeler wire 44 coupled to sensor D1 is touching a vulnerable plaque lesion 12, while the feeler wire 42, coupled to vibration sensor D2, is touching healthy vascular tissue 14. FIG. 5B represents the signal 46 obtained from each vibration sensor, D1 and D2. Detector signal DS1 corresponds with vibration sensor 1, and indicates the pronounced vibration of the lipid pool of the vulnerable plaque 12 caused by the pressure pulse 26. The presence of vulnerable plaque 12 can be diagnosed by comparing the vibration signal DS1 to the smaller vibration signal DS2 obtained from the vascular wall 14.

In another embodiment, the pressure pulse is produced by the contractions of the patient's heart, and a pressure pulse generator 18 is not needed. In those embodiments that include a pressure pulse generator 18, the pressure pulse is timed so that the measurements are made between contractions of the heart in order to minimize possible artifacts in the measurements due to the normal movement of the heart and vasculature.

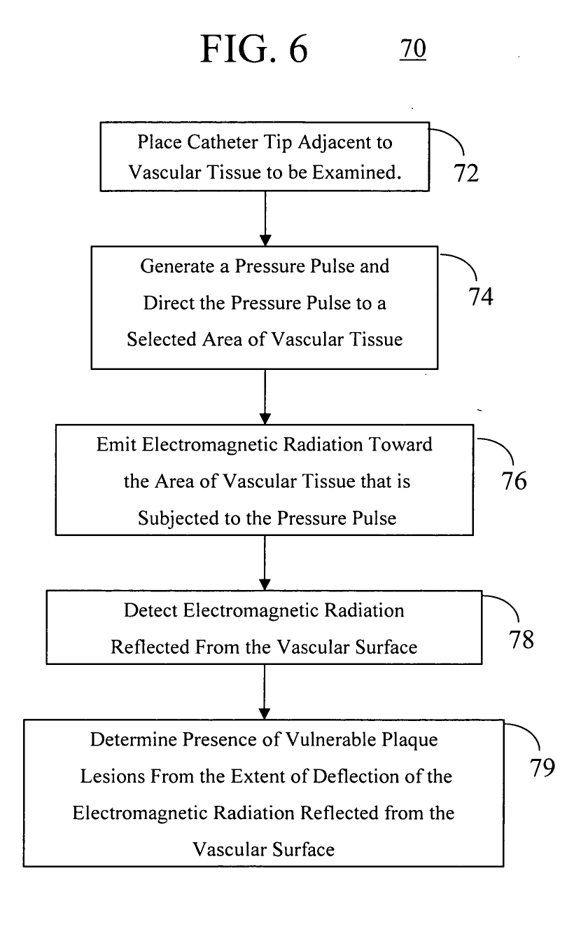

FIG. 6 is a schematic diagram of an embodiment of the invention comprising a method 70 for detecting vulnerable plaque lesions in blood vessels. The method 70 begins wherein a catheter having a pressure pulse generator, an electromagnetic radiation emitter, and an electromagnetic radiation sensor coupled to the catheter is placed adjacent to the area of the blood vessel that is to be examined for vulnerable plaque lesions (Block 72). The positioning of the catheter may be determined by known visualization methods such as fluoroscopy or ultrasound. Next, repeated, localized pressure pulses are generated in the vascular area near the distal tip of the catheter (Block 74). The pressure pulses are of sufficient magnitude to cause the vessel walls to distend slightly. The wall of the vessel is allowed to return to its relaxed state between pulses. Electromagnetic radiation is emitted toward the area of the vascular wall that is subjected to the pressure pulse (Block 76). Electromagnetic radiation is reflected from the surface of the vascular wall, and is detected by an electromagnetic radiation sensor located on the catheter (Block 78). The movement of the vessel wall and vulnerable plaque lesions that may be present both cause a deflection of the electromagnetic radiation reflected from the vessel wall, but each causes a distinctly different wave-like pattern in the amount of electromagnetic radiation received by the sensor. Because of their fluid nature, the vulnerable plaque lesions are more responsive to the pressure pulses, and produce a more pronounced wave-like pattern than the relatively stable vascular wall. The presence of vulnerable plaque lesions is determined from the extent of the deflection of the electromagnetic radiation reflected from the vascular surface (Block 79). In order to enhance the sensitivity of the method, the generation of the pressure pulse and the measurements of the deflection of the reflected electromagnetic radiation are timed so that the measurements are made between contractions of the heart. The changes in the amount of reflected electromagnetic radiation received by the sensor(s) may be converted to a signal and transmitted outside the patient's body to a processing unit, such as a computer.

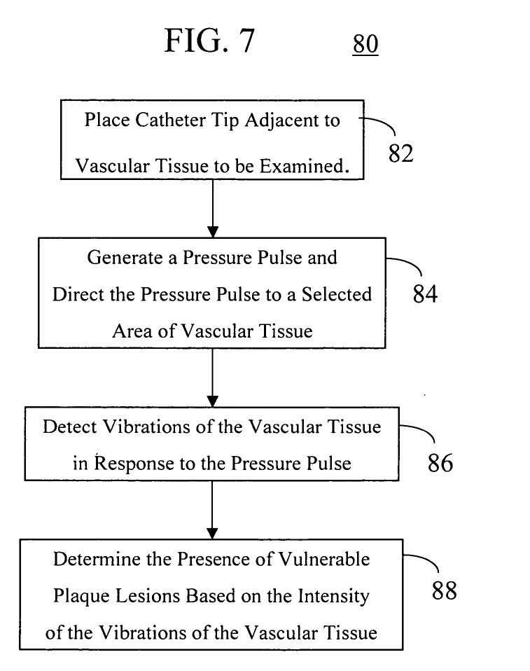

FIG. 7 is a schematic diagram of an embodiment of the invention comprising yet another method 80 for detecting vulnerable plaque lesions in blood vessels. The distal end of a catheter, having a vibration sensor operably coupled to the catheter, is placed adjacent to the vascular tissue that is to be examined for the presence of vulnerable plaque (Block 82). The catheter may also have a pressure pulse generator coupled to it. Next, a series of localized pressure pulses is generated (Block 84). The pressure pulses are of sufficient magnitude to cause the vessel walls to distend. The pressure pulses may result from the patient's heart contractions or they may be generated by the pressure pulse generator coupled to the catheter. In either case, the pressure pulses cause vibrations in the vascular tissue surrounding the distal tip of the catheter. These vibrations are received by the vibration sensor coupled to the catheter (Block 86). Due to their high lipid content, the vulnerable plaque lesions respond to the pressure pulses by producing larger vibrations than those produced by healthy vascular tissue. Therefore, the presence of vulnerable plaque lesions can be determined by measuring the intensity of the vibrations of the vascular tissue in response to the pressure pulses (Block 88). The information received by the vibration sensor is then converted into a signal, which may be transmitted from the patient's body to a processing unit, such as a computer.

While the embodiments according to the present invention are disclosed herein, various changes and modifications can be made without departing from the spirit and scope of the invention. For example, a variety of vascular locations may be examined using the present invention. The components of the device may be arranged in various configurations, and numerous modifications may be made to both the device and the method while providing effective vulnerable plaque detection consistent with the present invention. Furthermore the methods of the present invention may be combined with various medical procedures, depending on the condition and needs of the patient.

1. A method for detecting vulnerable plaque in a vessel, the method comprising:
sending a pressure pulse toward a vascular tissue;
emitting electromagnetic radiation toward the vascular tissue;
detecting a resulting electromagnetic energy from an interaction of the emitted electromagnetic energy on the vascular tissue subjected to the pressure pulse; and
determining at least one vulnerable plaque lesion in the vascular tissue based on the resulting energy.

2. The method of claim 1 wherein the pressure pulse is generated by a heart contraction.

3. The method of claim 1 wherein the pressure pulse is generated by a pressure pulse generator selected from a group consisting of a piezoelectric element, an electrostatic element, and an inflatable balloon.

4. The method of claim 1 wherein the electromagnetic radiation is selected from a group consisting of infrared radiation, visible light radiation, ultraviolet radiation, x-ray radiation, beta radiation, fluorescence radiation, laser light, microwave radiation, radio wave, and ultrasonic energy.

5. The method of claim 1 wherein detecting a vulnerable plaque lesion in the vascular tissue based on the resulting electromagnetic radiation comprises determining a variation in a plurality of resulting electromagnetic radiation measurements.

6. The method of claim 1 further comprising administering a drug to expand or contract the vascular tissue.

7. The method of claim 6 wherein a fibrous cap of the vulnerable plaque lesion has an increased or lessened tension based on the administered drug.

8. The method of claim 1 wherein the resulting electromagnetic radiation is detected by a receiver positioned on a catheter within a vessel.

9. The method of claim 8 wherein the receiver has a narrow angle of acceptance for the impinging electromagnetic radiation.

10. The method of claim 8 wherein the catheter further includes an expandable member, and the expandable member is expanded to stabilize the catheter within the vessel.

11. The method of claim 1 wherein the pulse is generated subsequent to a heart QRS wave impulse.

12. A method for detecting vulnerable plaque in a vessel, the method comprising:
sending a pressure pulse toward vascular tissue;
detecting a resulting vibration from an interaction of the of the pulse on the vascular tissue; and
determining at least one vulnerable plaque lesion in the vascular tissue based on the resulting vibration.

13. The method of claim 12 wherein the pressure pulse is generated by a heart contraction.

14. The method of claim 12 wherein the pressure pulse is generated by a pressure pulse generator selected from a group consisting of a piezoelectric element, an electrostatic element, and an expandable balloon.

15. The method of claim 12 wherein detecting the resulting vibration comprises receiving vibrations through feeler wires contacting a wall of the vessel.

16. A system for detecting vulnerable plaque in a vessel comprising:
a catheter;
a pressure pulse generator coupled to the catheter;
an electromagnetic radiation emitter coupled to the catheter, and
an electromagnetic radiation detector coupled to the catheter to receive reflected electromagnetic radiation that is directed at vascular tissue subjected to a pressure pulse.

17. The system of claim 16 wherein the pressure pulse generator is selected from a group consisting of a piezoelectric element, an electrostatic element, and an inflatable balloon.

18. The system of claim 16 wherein the electromagnetic radiation emitter is selected from a group consisting of an infrared radiation emitter, a visible radiation emitter, an ultraviolet radiation emitter, an x-ray radiation emitter, a beta radiation emitter, a fluorescence radiation emitter, a laser light emitter, a microwave radiation emitter, a radio wave emitter, and an ultrasonic energy emitter.

19. A system for detecting vulnerable plaque in a vessel comprising:
a catheter; and
a vibration sensing device coupled to the catheter to receive vibrations from vascular tissue.

20. The system of claim 19 further comprising:
a pressure pulse generating device coupled to the catheter.

21. The system of claim 19 wherein the vibration-sensing device comprises feeler wires that contact the vascular tissue.

22. A method for detecting vulnerable plaque lesions in a blood vessel comprising:
causing a localized change in pressure in the blood vessel;
measuring a motion of an internal surface of the blood vessel in response to the change in pressure; and
determining at least one vulnerable plaque lesion in the blood vessel based on a difference in motion between the surface of the vulnerable plaque and the surface of the healthy blood vessel.
